(19) United States
(12) Plant Patent Application Publication
(10) Pub. No.: US 2007/0033683 P1
(43) Pub. Date: Feb. 8, 2007

(54) Floribunda rose plant named 'Meinusian'

(75) Inventor: Alain A. Meilland, Antibes (FR)

Correspondence Address: BUCHANAN, INGERSOLL & ROONEY PC, POST OFFICE BOX 1404, ALEXANDRIA, VA, 22313-1404, US

(73) Assignee: CP DELAWARE, INC., West Grove, PA (US)

(21) Appl. No.: 11/545,545

(22) Filed: Oct. 11, 2006

Related U.S. Application Data
(63) Continuation of application No. 11/353,183, filed on Feb. 14, 2006.
Application No. 11/353,183 is a continuation of application No. 09/238,742, filed on Jan. 28, 1999, now abandoned.

(51) Int. Cl. A01H 5/00

(52) U.S. Cl. PLT/150

(57) ABSTRACT

A new and distinct variety of Floribunda rose plant is provided which abundantly forms on a substantially continuous basis attractive blossoms that are vermilion red in coloration. Such blossoms possess no fragrance. The plant exhibits a bushy growth habit, dark green and semi-glossy foliage, and very good disease resistance. The new variety is particularly well suited for providing colorful ornamentation in the landscape.

This is a Continuation of application Ser. No. 11/353,183, filed Feb. 14, 2006, which is a Continuation of application Ser. No. 09/238,742, filed Jan. 28, 1999.

### BOTANICAL/COMMERCIAL CLASSIFICATION

Rosa hydrida/Floribunda Rose Plant

### VARIETAL DENOMINATION

cv. ‘Meinusian’

### SUMMARY OF THE INVENTION

The new variety of Rosa hydrida Floribunda rose plant was created by artificial pollination wherein two parents were crossed which previously had been studied in the hope that they would contribute the desired characteristics. The female parent (i.e., the seed parent) of the new variety was the product of the cross of the ‘Meichanso’ variety (non-patented in the United States) and the ‘Meifolio’ variety (non-patented in the United States). The male parent (i.e., the pollen parent) was the ‘Korlima’ variety (non-patented in the United States). The ‘Meichanso’ variety sometimes is known as the ‘Parador’ variety. The ‘Korlima’ variety sometimes is known as the ‘Lilli Marleen’ variety. The parentage of the new variety can be summarized as follows:

(‘Meichanso’×‘Meifolio’)×‘Korlima’).

The seeds resulting from the above pollination were sown and small plants were obtained which were physically and biologically different from each other. Selective study resulted in the identification of a single plant of the new variety.

It was found that the new variety of Floribunda rose plant of the present invention possesses the following combination of characteristics:
(a) forms on an abundant and substantially continuous basis attractive double blossoms that are vermilion red in coloration,
(b) exhibits a bushy low and compact growth habit,
(c) forms attractive dark green semi-glossy foliage, and
(d) is particularly well suited for growing as ornamentation in parks and gardens.

The petals of fully open blossoms when viewed from above tend to be arranged in quartered clusters.

The new variety of the present invention readily can be distinguished from its ancestors. More specifically, the new variety can be readily differentiated from the ‘Meichanso’, ‘Meifolio’, and ‘Korlima’ varieties by a substantially greater number of petals and the resulting flower configuration (as illustrated).

The new variety well meets the needs of the horticultural industry and is particularly well suited for growing in the landscape.

The new variety has been found to undergo asexual propagation in France by a number of routes, including budding, grafting, and cuttage. Asexual propagation by the above-mentioned techniques in France has shown that the characteristics of the new variety are stable and are strictly transmissible by such asexual propagation from one generation to another.

The new variety has been named the ‘Meinusian’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were two years of age and were observed during June while budded on Rosa froebelli understock and growing outdoors at Le Cannet des Maures, Var, France. Dimensions in centimeters are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower in the course of opening;

FIG. 6—illustrates a specimen of an open flower—plan view—obverse;

FIG. 7—illustrates a specimen of an open flower—plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates a specimen of a leaf with three leaflets—plan view—upper surface;

FIG. 15—illustrates a specimen of a leaf with five leaflets—plan view—under surface; and

FIG. 16—illustrates a leaf with seven leaflets—plan view—upper surface.

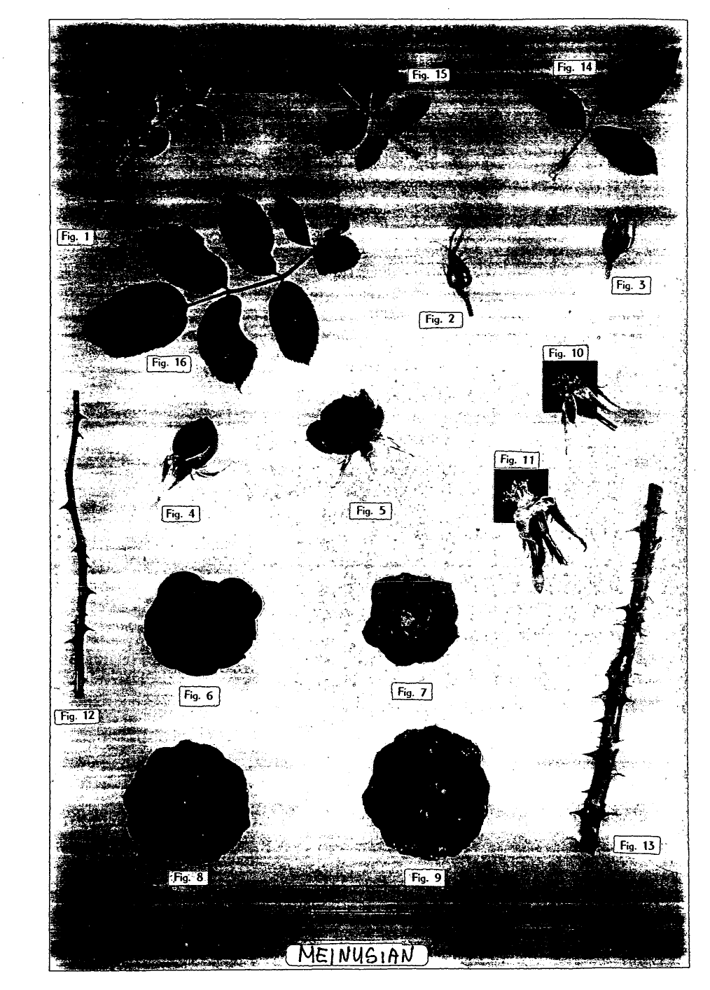

### DETAILED DESCRIPTION

The chart used in the identification of the colors is that of the Royal Horticultural Society (R.H.S. Colour Chart). The description is based on the observation of two year-old plants during June while budded on Rosa froebelli understock and growing outdoors at Le Cannet des Maures, Var, France. The coloration in common terms precedes reference to the chart in some instances.
Class: Floribunda.
Plant:
   Height.—approximately 70 to 80 cm. on average at the end of the growing season.
   Habit.—bushy.
Branches:
   Color.—young stems: near Yellow-Green Group 146A and stained with reddish coloration. adult wood: near Green Group 139B and stained with darker green of Green Group 139A.
   Thorns.—size: medium (as illustrated). quantity: numerous (as illustrated). color: reddish on young shoots and pinkish-brown on adult wood.
Leaves:
   Stipules.—adnate, pectinate, and moderately large.
   Petioles.—upper surface: striped reddish brown on young foliage and light green on adult foliage. under surface: light green in coloration and possess some prickles.
   Leaflets.—number: 3, 5 (most often) and 7. shape: elliptic. serration: simple and regular (as illustrated). texture: leathery. general appearance: dark green, and semi-glossy. color (young foliage): upper surface: near Yellow-Green Group 147A and widely suffused with reddish coloration. under surface: Yellow-Green Group 146A and very widely suffused with reddish coloration. color (adult foliage): upper surface: dark green, near Green Group 139A. under surface: near Green Group 137B.
Inflorescence:
   Number of flowers.—usually two to five flowers per stem.
   Peduncle.—light green with some acidulate prickles, and the length is approximately 5.5 cm. on average. The coloration is near Green Group 138B with some Brown Group 200C at maturity.
   Sepals.—upper surface: tomentose, greenish and lightly suffused with reddish coloration. The upper surface is near Green Group 138B and the under surface is near Green Group 138A. under surface: light green suffused with reddish coloration, and commonly without extensions.
   Buds.—shape: globular. size: large. length: approximately 3 cm. on average. color upon opening: upper surface: near Red Group 45B. under surface: near Red Group 45B suffused with Red Group 53A.
   Flower.—shape: cupped with a substantially flat head. diameter: commonly approximately 8 cm. on average. color (when opening begins): upper surface: near Red Group 43A. under surface: near Red Group 53C. color (when blooming): upper surface: near Red Group 43A. under surface: near Red Group 53C. color (at end of opening): upper surface: near Red Group 50A. under surface: near Red Group 53D. fragrance: none. lasting quality: very long on the plant. The blossoms commonly last approximately 15 days on the plant and approximately 5 days when cut and placed in a vase. petal number: commonly approximately 80 on average. petal shape: obtuse. stamen number: approximately 65 on average. anthers: pinkish-yellow in coloration. filaments: pinkish in coloration. pistils: approximately 50 on average. stigmas: fuchsine in coloration. styles: straw-colored receptacle: light green in coloration, smooth, and in longitudinal section in the shape of a pitcher.
Development:
   Vegetation.—strong.
   Blooming.—very abundant and substantially continuous.
   Resistance to diseases.—very good with respect to Botrytis, Powdery Mildew, and Downey Mildew.

I claim:

1. A new and distinct variety of Floribunda rose plant characterized by the following combination of characteristics:
(a) forms on an abundant and substantially continuous basis attractive double blossoms that are vermilion red in coloration,
(b) exhibits a bushy low and compact growth habit,
(c) forms attractive dark green semi-glossy foliage, and
(d) is particularly well suited for growing in parks and gardens; substantially as herein shown and described.
